(19) United States
(12) Patent Application Publication
(10) Pub. No.: US 2026/0024665 A1
(43) Pub. Date: Jan. 22, 2026

(54) Risk Score and Classification System for Prevention of Complications in Plastic Surgery

(71) Applicant: AIRA10 MEDICAL LLC, Albuquerque, NM (US)

(72) Inventors: Williams Erik Bukret, Buenos Aires (AR); Karina Alejandra Pesce, Buenos Aires (AR)

(21) Appl. No.: 18/887,655

(22) Filed: Sep. 17, 2024

Related U.S. Application Data
(63) Continuation-in-part of application No. 18/777,035, filed on Jul. 18, 2024.

(51) Int. Cl. G16H 50/30; G16H 10/20; G16H 10/60; G16H 50/20

(52) U.S. Cl. CPC G16H 50/30; G16H 10/20; G16H 10/60; G16H 50/20

(57) ABSTRACT

A real-time method for risk assessment and prediction of complications in plastic surgery. It includes a scoring method that classifies patients in distinct levels or risk-groups according to a risk score. According to the variables present in each individual, the algorithm estimates a risk factor for that particular data set provided by the patient through a questionnaire, calculates a risk score, classifies each patient in a risk group, and displays results in different electronic devices and personalized apps.

### CROSS-REFERENCE TO RELATED APPLICATION

This application is a Continuation in Part of U.S. Provisional patent application Ser. No. 17/888,035, filed Aug. 15, 2022, which is incorporated by reference herein in its entirety.

### TECHNICAL FIELD

This invention relates generally to a multifactor risk prevention and scoring system and method for surgical procedures and, more particularly, to a computerized system and method for predicting complications after cosmetic surgeries based on a multifactorial analysis, allowing patients to be classified into different risk groups.

### BACKGROUND OF THE INVENTION

Prevention of complications is one of the most important concerns of plastic surgeons since it implies reducing the costs of hospitalizations, treatments, reoperations and also increasing the degree of patient satisfaction. Thus, it is necessary to have an efficient universal system that serves for risk analysis and the prevention of complications. Currently, the recommendations of the expert consensus guidelines are used for prevention and there is no model that can predict complications in real-time, taking into account the risk factors of each patient. However, these proposed norms and parameters vary according to the authors and should be updated based on evidence. Furthermore, these guidelines are not practical when evaluating patients because they often do not apply to particular cases, which makes it difficult to adequately classify patients according to their risk.

According to the guide of recommendations of the American Society of Plastic Surgery of the year 2011, which is still in force, high-risk patients are classified as those with a body mass index≥35, age≥50 years, liposuction greater than 5000 ml, and combined surgeries particularly abdominoplasty with liposuction. Later in 2018, Dr. Rod Rohrich suggested a change in the classification parameters of high-risk patients: body mass index≥30, age≥40 years, liposuction greater than 3 liters (Table 1).

TABLE 1
Recommendations for prevention of complications.

Recommendations

| | ASPS ¹ | Rohrich, R² |
|---|---|---|
| Age | >50 | >40 |
| BMI | >40 | >30 |
| Lipoaspirate | >5000 ml | >3000 ml |
| Combined surgeries | Especially | |
| | abdominoplasty | |
| | with liposuction | |

BMI, body mass index. ASPS American Society of Plastic Surgeons; ASPS Patient Safety Committee. Pathways to preventing adverse events in ambulatory surgery in 2011. Rohrich R J, Mendez B M, Afrooz P N. An update on the safety and efficacy of outpatient plastic surgery: A review of 26,032 consecutive cases. Plast Reconstr Surg. 2018, (4): 902-908.

However, at present, there is no availability of a risk prediction model that adjusts to variations in the mentioned risk factors. For this reason, providing a method and system for predicting complications after plastic surgery based on a classification of patients in different risk groups in consideration of a multiplicity of risk factors and their variations fulfills a long felt yet unresolved need, substantially advancing the field.

### SUMMARY OF THE INVENTION

The main object of the present invention is to provide a computing system for establishing a predictive score for the risk of complications after plastic surgery, and for classifying patients into different risk groups. For this reason, a multifactorial correlation analysis was performed between risk factors and complications to generate an evidence-based multifactorial classification system.

The Risk Score and Classification System for the prevention of complications during surgery relies on a dynamic, evidence-based approach. Through a real-time assessment of the patient's medical history and specific risk factors such as BMI, Caprini Score, smoking habits, and age, this system can predict the risk of complications before surgery.

The classification process is divided into Low, Moderate, and High Risk categories:
Low Risk: The patient is considered fit for surgery with minimal intervention, proceeding with basic health advice.
Moderate Risk: Surgery can proceed, but only after addressing specific risk factors (e.g., weight management or blood pressure control).
High Risk: Surgery is delayed until the significant risk factors are managed. Intensive treatments and reassessments are recommended before considering any surgical procedure.

These classifications not only streamline the decision-making process between the patient and doctor but also personalize the treatment recommendations based on the patient's individual profile. This system assists surgeons in optimizing safety and preventing complications, ensuring better patient outcomes. It also helps patients proactively understand their own health conditions and risk factors, and how to modify them as a general preventive measure, particularly important when considering surgery. This empowers patients, encourages active healthcare participation, and reduces cost burdens for all stakeholders. Patients can independently access their own risk assessment online, without the need for a surgeon.

In preliminary studies, the relationship and relative risk were established between several independent risk factors and complications. Consequently, it is still necessary to establish the role of some risk factors such as smoking, sex, combined surgeries, as well as the combination of risk factors in a predictive model to establish an efficient risk analysis model. Thus, the present invention considers the risk factors of each patient individually, classifies patients according to their risk of suffering complications through a scoring system, thus determining the probabilities of suffering the most common complications after plastic surgery. Moreover, the proposed system classifies patients in real-time into different risk groups based on their risk score. Another advantage of the system is that it allows personalized recommendations to reduce risk, improving the eligibility of the patient and the appropriate surgical environment according to their risk. Overall, the risk assessment system does not intend to replace plastic surgeon evaluation but augment it by facilitating the collection of data, avoiding errors, optimizing time, and reducing cost. Another advantage is that the information is always available and displayed on multiple devices.

A computing system for establishing a predictive score for the risk of complications after plastic surgery in accordance with the present invention, is achieved by combining some or all of the following aspects:
a) A computer or electronic device comprising a processor and a memory and connected to internet
b) An online evaluation form accessible through a patient interface that the patient can access through a computer or electronic device comprising a processor and a memory and connected to the internet. This form includes input-form-fields to enter patient information, medical antecedents, and risk factors. All the data needed to calculate the Caprini Score of the patient should be recollected in this step as well as the cigarette consumption, BMI (or height and weight) and age of the patient. The system includes built-in data validation mechanisms to ensure that patient-entered information is cross-checked against medical standards. Alerts will notify patients and physicians of any inconsistencies in the data, minimizing the risk of misclassification or errors in the risk assessment
c) A database in which the information from point b is collected, stored, and from which it can be retrieved as required
d) An automated risk-score calculation algorithm based on the information stored on database c, using the following formula to calculate the Risk Score of the patients:

$$Risk Score = {W}_{1} {f}_{1} + {W}_{2} {f}_{2} + {W}_{3} {f}_{3} + {W}_{4} {f}_{4}$$

Where W1, W2, W3, and W4 are variables of the formula and f1, f2, f3, and f4 are factors of the formula, defined as:
W₁ is the age of the patient, W₂ is the BMI of the patient, W₃ is the Caprini Score of the patient, W₄ is the number of cigarettes the patient smokes per week, f₁ is 100\hat{ }(−3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more.
e) A risk classification algorithm to assign a Risk group to the patients based on the Risk Score calculated in point d, as low risk (1 to <1.2 points), moderate risk (≥1.2 to <1.4 points), and high risk (≥1.4 points) of complications.
f) A deployment of information process to display the results through a Physician interface in a computer or electronic device comprising a processor and a memory and connected to the Internet. To accommodate a range of users, the system includes a simplified interface option that presents key risk assessment data in a clear, non-technical format. Additionally, the app features a detailed user guide and context-sensitive help system to explain complex algorithms in layman's terms, ensuring ease of use for all medical professionals. The system features a simplified user interface designed for both surgeons and patients. This intuitive design ensures that complex medical data and recommendations are displayed in an easily understandable format. The interface is optimized for non-technical users, providing a clear and user-friendly experience, allowing patients to easily access their own risk scores and recommendations.
g) Advanced statistical methods to identify significant risk factors and predict post-surgical complications with high accuracy, ensuring reliable and personalized recommendations.
h) A system for ensuring the security and privacy of patient data, in compliance with global health data protection regulations such as the General Data Protection Regulation (GDPR) and the Health Insurance Portability and Accountability Act (HIPAA). The system integrates data encryption both during transmission and at rest, safeguarding patient data from unauthorized access.
i) A reassessment mechanism to keep the information in the database up to date, and
j) The computing system is embodied in a computer having a processor, a memory, a screen, and internet access.

In the adaptive mode, the system fine-tunes the variables by collecting clinical data and performing statistical analysis. The system uses Pearson's correlation coefficient test and p-value analysis to identify risk factors with statistically significant correlations to post-surgical complications. By performing Pearson's correlation and p-value tests between the risk score and individual risk factors, the system determines which factors act as independent predictors of complications.

Based on the results of this multifactorial correlation analysis, the system establishes a predictive scoring system that is continuously refined through coefficient of multiple correlation testing. This predictive scoring system can either operate with the validated fixed coefficients or, when in adaptive mode, continuously improve through machine learning algorithms, particularly using a Support Vector Classification (SVC) model.

The SVC model analyzes new clinical data and updates the values of the variables and coefficients in the formula to enhance predictive accuracy. The system ensures ongoing validation of the scoring system as new data is integrated, confirming that the risk factors remain clinically significant and accurate over time.

This dual capability-using either the static, validated model or continuous machine learning-based updates-allows the system to be flexible and adaptable to both clinical environments that prefer a predictable, fixed formula and those that wish to leverage continuous improvement based on evolving clinical data.

Additionally, a prospective clinical trial (Registration No. NCT06507384) titled “Prospective Observational Study of an Artificial Intelligence Risk Assessment Model for Complication Prevention in Plastic Surgery” was conducted, evaluating 3,347 patients. The AI model successfully stratified patients into low, moderate, and high-risk categories. Of the 74 surgical patients, 9.46% experienced complications, primarily within the high-risk group (relative risk 6.73). Significant correlations between complications and key risk factors further validate the model's predictive accuracy and efficacy in real-world clinical settings, confirming its relevance and reliability.

Furthermore, multi-layered access control mechanisms are employed to ensure that only authorized personnel can access sensitive information. These controls include user authentication, role-based access, and audit trails, which track access and modifications to the data, ensuring accountability and security.

By complying with GDPR and HIPAA regulations, the system guarantees the highest standards for data security and patient privacy. Clinics and hospitals operating in multiple jurisdictions can confidently utilize the system, knowing it meets legal requirements for protecting patient information. These built-in security measures prevent data breaches, minimize the risk of misuse, and provide patients with confidence in the confidentiality of their medical information. The system's modular data security measures are designed to accommodate clinics of all sizes, allowing them to scale security features based on their size, available resources, and jurisdictional requirements. Clinics in regions with stricter regulatory frameworks can implement advanced encryption, multi-layered access control, and secure cloud storage solutions, while smaller clinics can opt for streamlined data protection options that still meet essential GDPR and HIPAA compliance standards.

A method to establish a scoring system and classification into risk groups for predicting the risk of complications after plastic surgeries is disclosed, which includes the following steps:
(1) Collecting clinical data from the patients, including all the information needed to calculate the Caprini Score of the patient as well as the cigarette consumption, BMI (or height and weight) and age of the patient, through an online evaluation form;
(2) Storing the data from step 1 on a database;
(3) Calculating the Caprini Score of the Patient and the BMI based on height and weight. A Person of Ordinary Skill in the art is aware of how to calculate a Caprini Score. However, to avoid any possible misinterpretation, the article “Pannucci, C. et.al (2016). Benefits and Risks of Prophylaxis for Deep Venous Thrombosis and Pulmonary Embolus in Plastic Surgery. Plastic and Reconstructive Surgery, 137 (2), 709-730. doi: 10.1097/01.prs.0000475790.54231.28,” is attached to the submission of the present application and hereby incorporated by reference in its entirety;
(4) Calculating the Risk Score of the patient using the formula:

“Risk score=W₁ f₁+W₂f₂+W₃f₃+W₄f₄₌₁-2.5” to the information collected from the patient, where W₁ is the age of the patient, W₂ is the BMI of the patient, W₃ is the Caprini Score of the patient, W₄ is the number of cigarettes the patient smokes per week, f₁ is 100\hat{ }(−3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more:
(5) Classifying the Patients in Risk Groups according to the algorithm:
Risk of complications: low risk when the Risk score is (1 to <1.2 points), moderate risk when the Risk score is (>1.2 to <1.4 points), and high risk when the Risk score is (≥1.4 points);
(6) Displaying the Risk Score calculated on step 4 and the risk group calculated on step 5, together with the clinical data collected on step 1, though a Physician's interface in a computer or electronic device comprising a processor and a memory and connected to the Internet;
and
(7) Defining a concrete treatment and course of action depending on the Risk Score calculated on step 4 and the risk group calculated on step 5, together with the clinical data collected on step 1, with use of an Algorithm Recommendations Table.

Another key advantage of the present invention is the significant improvement in workflow efficiency. Traditional risk assessment methods, which involve gathering patient data, manually calculating scores (e.g., BMI, Caprini Score), and determining risk factors, can take 20 to 60 minutes. This process often requires clinicians to spend considerable time on manual calculations and reviewing patient histories.

In contrast, the present system automates the entire process, allowing the patient's risk profile to be calculated in approximately one minute. Through the use of an online evaluation form and an automated risk-score algorithm, the system generates immediate results, providing both the patient and the physician with a comprehensive understanding of the patient's risk classification even before the consultation begins.

This reduction in time greatly optimizes clinical workflows, allowing physicians to focus more on treatment planning and patient interaction, rather than administrative tasks. Additionally, patients can access their own risk assessments online before meeting with their doctor, promoting a more informed and proactive approach to surgical decisions. The time-saving feature also allows clinics to serve more patients efficiently, improving resource allocation, safety, and accuracy.

The system is integrated into a user-friendly app designed for both surgeons and patients, making it accessible and practical in various clinical settings. The app allows surgeons to input patient data, view risk assessments, and receive personalized recommendations to optimize patient outcomes and safety. Meanwhile, patients can use the app to access an online risk self-assessment tool, which helps them independently evaluate their risk factors before consulting with a surgeon. This dual access ensures that both parties are informed and prepared before making surgical decisions.

The system is not limited to plastic surgery but can be easily adapted to other surgical specialties, making it a versatile tool across various medical fields. By adjusting the input variables and coefficients, the system can be customized for orthopedic surgeries, cardiovascular procedures, general surgeries, and more. This flexibility allows the system to be used across multiple disciplines, enhancing its value and applicability in broader clinical contexts.

A key advantage of the system is the direct access it provides to patients through the app's online risk self-assessment tool. This feature benefits the general population considering elective surgeries, enabling them to independently assess their personal risk factors before consulting with a surgeon. By gaining early insights into their risk profiles, patients are empowered to take proactive measures, such as managing weight or quitting smoking, to reduce their risk and improve surgical outcomes.

This self-assessment tool not only enhances patient engagement but also allows healthcare providers to offer a personalized and informed approach to treatment planning, ultimately improving the safety and success of elective surgeries.

To accommodate clinics with limited computational resources, the system offers a lighter version. This version reduces the computational load while maintaining core functionalities, making it suitable for smaller clinics and practices. The lighter version ensures that even clinics without advanced technological infrastructure can benefit from the system's risk assessment capabilities.

### BRIEF DESCRIPTION OF THE DRAWINGS

FIG. 1 is a conceptual flow diagram of an embodiment of a system for establishing a predictive score for the risk of complications after plastic surgery in accordance with the present invention.

FIG. 2 is a conceptual flow diagram of the data management process in an embodiment of a system for establishing a predictive score for the risk of complications after plastic surgery in accordance with the present invention.

FIG. 3 is a table containing data collected from patients in an embodiment of a system for establishing a predictive score for the risk of complications after plastic surgery in accordance with the present invention.

FIG. 4 is a flow chart which provides a step-by-step guide, leading the user through the decision-making process from calculating risk to determining appropriate surgeries and whether combinations are allowed.

### DETAILED DESCRIPTION AND BEST MODE OF IMPLEMENTATION

Disclosed is a system for establishing a predictive score for the risk of complications after plastic surgery, and for classifying patients into different risk groups. A research study was performed to evaluate the risk factors predictive of complications and validate the new assessment system by a machine learning process. In it, a retrospective analysis was performed with 372 patients operated on by the author between 2015 and 2020 to ensure the consistency of the results for internal validation. Pearson's correlation test was used for the analysis of risk factors and complications. The difference between the mean of the risk scores of the three risk groups was assessed using one-way analysis of variance (ANOVA). For the analysis of the incidence of risk factors and relative risk of complications after cosmetic surgeries, systematic reviews and meta-analyzes, multicentric analyzes, and evidence-based research studies classified as type 1 and 2 were included, with more than 1000 cases in the last 5 years. The incidence of independent factors that exhibited a statistically significant positive correlation (p<0.05) between risk factors and complications and the relative risk (RR) was analyzed. Each risk factor was assigned a weighted coefficient (f) to stratify the risk of complications. Subsequently, a weighted sum method with the relevant risk factors (W) was used to create a risk scale from 1 to 2.5. The minimum risk was assigned the number 1 to obtain a minimum multiplication factor since any surgery involves some risk that is different from 0.

The clinical data obtained from the patients, including the independent risk factors, are personal data of the patient, age, sex, weight, height, smoking, previous surgeries, medical history, pathological history, requested surgery, and body measurements. The indexes used by the algorithm are calculated by the system from the data supplied by the patients: body mass index, Caprini score, body dysmorphic syndrome detection test, etc. Then the model calculates the risk score according to the data, classifying to which risk groups each patient belongs using the mentioned formula.

A simplified scoring system is established based on the weighted sum model. The risk score is a weighted sum method assisted by a narrow AI, expressed mathematically by the following formula:

$$Risk Score = {W}_{1} {f}_{1} + {W}_{2} {f}_{2} + {W}_{3} {f}_{3} + {W}_{4} {f}_{4} = 1 - 2.5$$

where W₁ is the age of the patient, W₂ is the BMI of the patient, W₃ is the Caprini Score of the patient, W₄ is the number of cigarettes the patient smokes per week, f₁ is 100\hat{ }(−3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more.

The system automatically classifies patients into one of the three risk groups according to their scores, such as A=low risk (1 to <1.2 points), B=moderate risk (>1.2 to <1.4 points) and C=high risk (≥1.4 points) of complications.

TABLE 2
Risk score and risk group classification

| Risk groups | Score | Risk |
|---|---|---|
| A | 1 to <1.2 | Low |
| B | ≥1.2 to <1.4 | Moderate |
| C | ≥1.4 to <2.5 | High |

The classification and scoring model were validated through machine learning in Python language using a support vector classification process (SVC). The predictive algorithm included the following risk factors are selected based on their relative importance and statistical significance (p<0.01): age, BMI, Caprini score, and smoking. The set of 372 data cases was divided into 2 samples. A training sample (0.80) and a test sample (0.20) with a random seed of 100 cases. Patients with missing data were excluded from the data set. All procedures performed in this study were performed in accordance with the Declaration of Helsinki. All patient information was disidentified and retrospective.

The frequency of cases, complications, odds ratios (OR) and the mean risk score in each risk group were as follows: low risk, 215 cases (9 complications, mean=1.04, odds ratio [OR]=0, 04, 99% confidence interval [CI]=1.045-1.035); moderate risk, 113 cases (9 complications, mean=1.27, OR=0.09, 99% CI=1.277-1.263); and high risk, 44 cases (10 complications, mean=1.67, OR=0.29, 99% CI=1.673-1.627). The difference between the means of the risk groups assessed with ANOVA was the following: f-value=1461.2, p-value=4.54828884e-176 (Table 3).

TABLE 3
Frequency of cases, complications, mean
and Odd Ratio per group (n = 372)

| Risk group | Cases | Complications | Mean | OR |
|---|---|---|---|---|
| Low | 215 | 9 | 1.04 | 0.04 |
| Moderate | 113 | 9 | 1.27 | 0.09 |
| High | 44 | 10 | 1.65 | 0.29 |

OR odd ratio

The measures used to evaluate the performance of the model were the following: accuracy score, precision (positive predictive value), recall (sensitivity), and harmonic mean of precision and recovery (f₁ score or Sørensen-Dice coefficient).

The accuracy score for SVC prediction was 100% in the training sample and 97.3% in the test sample. The positive predictive value of the method was 0.98, the sensitivity showed a weighted average of 0.97 and the f₁ score showed a weighted average of 0.97 (Table 4).

TABLE 4
Model Performance per Risk Group

| | Precision | Recall | f1-score | Support |
|---|---|---|---|---|
| Low | 1.00 | 0.83 | 0.91 | 6 |
| Moderate | 1.00 | 0.98 | 0.99 | 48 |
| High | 0.91 | 1.00 | 0.95 | 21 |
| Accuracy | | | 0.97 | 75 |
| Weighted avg | 0.98 | 0.97 | 0.97 | 75 |

Precision, positive predictive value; Recall, sensitivity; F1-score, harmonic mean of the precision and recall; avg, average

A cloud data collection system comprises an online form, a database, a spreadsheet for data collection and analysis, and a result display system for several applications. The data analysis system can calculate the risk of complications and classify patients into different risk groups according to their score obtained based on the information provided by the data entry system, and the visualization system will display the results in different custom user interfaces (UI). Compared with the expert committee recommendation guidelines used so far, the present invention selected for the first time the independent risk predictors of complications after plastic surgery used Pearson's correlation coefficients to assign values to each risk factor, establish a risk prediction scoring system, and classified patients into risk groups. The performance score of the method evaluated by the accuracy score yields a value of 97.3%, which exhibits excellent predictive capacity. This system is easy to implement in daily practice, allowing for the first time a personalized evaluation in real-time, which allows improving the recommendation and education of patients to correct risk factors and choose the most appropriate surgical setting according to the risk of each patient.

To understand in a comparative way the difference in the probability of occurrence of complications in the different risk groups, we calculated the relative difference between groups. The relative difference in complications was 6.7 times greater in the high-risk group (RR, 0.29) and 2 times greater in the moderate-risk group (RR, 0.09) than in the low-risk group (RR, 0.04) (Table 5).

TABLE 5
Frequency of complications, relative risk,
and relative difference per group (n = 372)

| Risk group | Cases | Complications | Mean | RR | RD |
|---|---|---|---|---|---|
| Low | 215 | 9 | 1.04 | 0.04 | |
| Moderate | 113 | 9 | 1.27 | 0.09 | 2.0 |
| High | 44 | 10 | 1.65 | 0.29 | 6.7 |

The variables selected to be included in the predictive model are statistically significant variables. The correlation table of significant variables is as follows (Table 6). Smoking was also included in the predictive model based on evidence and expert recommendations

TABLE 6
Correlations of risk score with risk factors

| | Risk score | BMI | Age | Caprini score |
|---|---|---|---|---|
| Risk score | 1.00 | 0.99 | 0.97 | 0.98 |
| BMI | 0.99 | 1.00 | 0.94 | 0.95 |
| Age | 0.97 | 0.94 | 1.00 | 0.99 |
| Caprini score | 0.98 | 0.95 | 0.99 | 1.00 |

### Establishment of a Scoring System to Predict Complications

Based on the results of the multivariate analysis, a prediction model for complications was established. The formula is as follows:

$$Risk Score = W 1 f 1 + W 2 f 2 + W 3 f 3 + W 4 f 4 = 1 - 2.5$$

According to the data entered into the system, the assignment of the coefficients used in the algorithm will be (Table 7):

TABLE 7
Assignment of coefficients

| | Coefficients (f) |
|---|---|
| f1 | No/[100\hat{ }3] |
| f2 | ≥25 = 0.2 ≥30 = 0.4 |
| f3 | No * 0.245 |
| f4 | <7 = 0.1 ≥7 = 0.3 |

According to the scoring system, patients are classified into risk groups according to their scores, from which risk-adjusted recommendations are made. The relative risk of complication is significantly different in each group as was demonstrated by the ANOVA score and relative difference. Table 8.

TABLE 8
Risk group and recommendations according to score

| Risk group | Score | Risk | Recommendation |
|---|---|---|---|
| A | 1 to <1.2 | Low | In conditions |
| B | ≥1.2 to <1.4 | Moderate | Defer surgery |
| C | ≥1.4 to <2.5 | High | Defer surgery |

### The Predictive Value of the Model

For the validation of the predictive model with supervised machine learning, a database of 372 cases was used, which was divided into training (0.8) test (0.20) with a random seed of 100 cases. The performance metric of the chosen model was the accuracy score, the average accuracy between groups was 0.973, which indicates that the scoring system has a great predictive capacity, especially when the score is ≥1.2, since in the moderate and high-risk groups the accuracy reached 100% (Table 9).

TABLE 9
Model Performance per Risk Group

| | Precision | Recall | f1-score | Support |
|---|---|---|---|---|
| Low | 1.00 | 0.83 | 0.91 | 6 |
| Moderate | 1.00 | 0.98 | 0.99 | 48 |
| High | 0.91 | 1.00 | 0.95 | 21 |
| Accuracy | | | 0.97 | 75 |
| Weighted avg | 0.98 | 0.97 | 0.97 | 75 |

Precision, positive predictive value;
Recall, sensitivity;
F1-score, harmonic mean of the precision and recall;
avg, average

Additionally, a risk-adjusted price (RAP) can be added to the system, to measure a surgery price after taking into account the degree of risk given by the risk score. Therefore, RAP risk-adjusted price is a number that varies linked to the risk score and will change according to the case. Mathematically is explained as a multiplier of the risk factor by the surgery price: E.g. (surgery price) times (risk factor)=USD 2000×1.03=2060. The surgery prices must be provided by the user as a price list, so the app will take that information to calculate the RAP for each patient. The RAP is displayed in another column of the list, same as all other information for each patient. The purpose of risk-adjusted prices is to help doctors cover losses for future re-operations, treatment costs, and legal expenses associated with a higher risk of complications and at the same time to encourage patients to take action to reduce risk modifying risk factors before surgery, in other words, to actively participate in prevention.

To further enhance the decision-making process, the disclosed Risk Score and Classification System for Prevention of Complications in Plastic Surgery not only provides personalized recommendations based on the patient's risk profile but also determines whether surgery should proceed based on these recommendations. This final decision-making step is crucial, as it integrates all risk factors and recommendations to make a clear, data-driven decision on whether the surgery is advisable at the time of assessment.

### Key Components:

1. Risk Classification: The patient is classified into Low, Moderate, or High Risk based on their calculated risk score.
2. Personalized Recommendations: The algorithm generates specific recommendations tailored to the patient's risk category, addressing any identified risk factors.
3. Final Decision: Beyond providing recommendations, the system makes a clear determination on whether the patient is fit to proceed with surgery:
   Low Risk: Typically, surgery is recommended to proceed with minimal precautions.
   Moderate Risk: Surgery may proceed after addressing specific risks; however, certain cases may require further evaluation.
   High Risk: Surgery is often advised to be delayed until significant risks are mitigated, with a strong focus on intensive risk management.

Below is a table that incorporates the decision-making process alongside the risk classification and personalized recommendations:

TABLE 10
Algorithm Recommendations Table

| Risk Score & | | | Logic | | Final |
|---|---|---|---|---|---|
| Classification | Condition | Criteria | Conditions | Recommendations | Decision |
| A - Low Risk (1 | No risk | N/A | AND condition: | No specific | Surgery |
| to <1.2) | factors | | No | recommendations; | Proceeded |
| | present | | recommendations | patient is fit for | (Yes) |
| | | | necessary | surgery with | |
| | | | | general health | |
| | | | | maintenance only. | |
| A - Low Risk (1 | Specific risk | Example: | AND condition: | Quit smoking. | Surgery |
| to <1.2) | factor | Smoker | Address specific | Consider nicotine | Proceeded |
| | | | risk factor | replacement | (Yes) |
| | | | | therapy. | |
| B - Moderate Risk | Blood | High blood | AND/OR | Monitor BP daily | Surgery |
| (≥1.2 to <1.4) | Pressure | pressure | conditions: High | and consult | Proceeded |
| | Control | | BP and/or other | cardiologist. | (Conditional |
| | | | risks | | Yes) |
| B- Moderate Risk | Weight | BMI ≥25.1 | AND/OR | Recommended | Surgery |
| (≥1.2 to <1.4) | Management | | conditions: High | weight: | Proceeded |
| | | | BMI and/or | [Calculated | (Conditional |
| | | | other risks | Weight]. Adjust | Yes) |
| | | | | exercise plan and | |
| | | | | diet with | |
| | | | | nutritionist. | |
| C - High Risk | Ergometry | BMI >28 OR | AND/OR | Request | Surgery |
| (≥1.4) | and Doppler | hereditary/family | conditions: | ergometry and | Delayed |
| | for | diseases | Multiple risk | Doppler | (No) |
| | Cardiovascular | (e.g., | factors present | ultrasound of | |
| | Risk | thrombosis, | | lower limbs. | |
| | | stroke, | | Assess | |
| | | vasculopathies) | | cardiovascular | |
| | | OR high blood | | health, especially | |
| | | pressure AND | | for patients with | |
| | | age ≥45 OR | | multiple risk | |
| | | any | | factors. | |
| | | combination of | | | |
| | | these conditions | | | |
| C - High Risk | Coagulation | Diagnosed | AND/OR | Request | Surgery |
| (≥1.4) | Disorders | coagulation or | conditions: High | extended | Delayed |
| | | bleeding issues | Caprini score | laboratory | (No) |
| | | (e.g., factor | and/or other | analysis and | |
| | | deficiencies) | risks | consult | |
| | | | | Hematology. | |
| | | | | Ensure all | |
| | | | | necessary | |
| | | | | coagulation | |
| | | | | parameters are | |
| | | | | checked. If | |
| | | | | Caprini Score >8, | |
| | | | | order additional | |
| | | | | tests listed below. | |
| C - High Risk | Further Tests | Caprini | AND/OR | Consult with | Surgery |
| (≥1.4) | and | Score >8 | conditions: High | endocrinologist | Delayed |
| | Consultations | | Caprini score | and hematologist. | (No) |
| | | | | Order the | |
| | | | | following labs: t3, | |
| | | | | t4, TSH, | |
| | | | | Homocysteine, | |
| | | | | Factor V Leiden, | |
| | | | | Prothrombin | |
| | | | | 20210A, Lupus | |
| | | | | Anticoagulant | |
| | | | | (LA) | |
| | | | | Anticardiolipin | |
| | | | | antibodies. | |
| A, B, C - | Thyroid and | Hypothyroidism, | AND conditions: | Consult with | Surgery |
| Low/Moderate/High | Endocrinology | Hyperthyroidism, | Based on | endocrinologist. | Proceeded |
| Risk | Management | or BMI >31 | thyroid and BMI | Monitor thyroid | (Conditional |
| (1 to <2.5) | | | levels | levels regularly. | Yes) |
| A, B, C - | Nutritional | BMI <18.5 | AND conditions: | Request | Surgery |
| Low/Moderate/High | Guidance | | Low BMI and | proteinogram, | Proceeded |
| Risk | | | possible ED | increase weight, | (Conditional |
| (1 to <2.5) | | | | rule out eating | Yes) |
| | | | | disorders. Consult | |
| | | | | with nutritionist | |
| | | | | and psychiatrist. | |
| B, C - | Bariatric | BMI >31 | AND/OR | Consult with | Surgery |
| Moderate/High | Surgery | | conditions: High | bariatric surgery. | Delayed |
| Risk (≥1.2 to <2.5) | Referral | | BMI and/or | Consider surgical | (No) |
| | | | other risks | options if | |
| | | | | necessary. | |

Abbreviations:
BMI: Body Mass Index,
BP: Blood Pressure

### Explanation of Logic

1. Risk Classification: Patients are classified into three risk categories based on their calculated risk score:
   Low Risk (1 to <1.2): Patients in this category either have no significant risk factors or have manageable ones. If no specific risk factors are present, no special recommendations are needed. If there are specific risk factors (e.g., smoking), targeted recommendations, such as smoking cessation, are provided to address those risks.
   Moderate Risk (>1.2 to <1.4): This category includes patients with moderate risks, such as slightly elevated blood pressure or BMI. The recommendations focus on managing these risks, for example, daily blood pressure monitoring or weight management interventions.
   High Risk (>1.4): Patients in this category have significant risk factors or multiple risk factors, such as high BMI combined with family history of cardiovascular diseases. The recommendations are more intensive and may include further testing, specialist consultations, and specific interventions to mitigate the high risks.
2. Algorithm Logic:
   The algorithm uses a combination of “AND” and “OR” conditions to evaluate each patient's risk factors and determine the appropriate recommendations.
   AND condition: Used when all specified criteria must be met to trigger a recommendation (e.g., presence of high blood pressure and age≥45).
   OR condition: Used when any one of several criteria can trigger a recommendation (e.g., either high BMI or hereditary disease can prompt a cardiovascular evaluation).
   Combined Conditions: In many cases, both “AND” and “OR” conditions are used together to refine the recommendations based on multiple factors.
3. Personalized Recommendations:
   Each patient's risk score and classification determine the specific recommendations provided. These are designed to prevent complications by addressing the unique risk factors of the patient, ensuring they are adequately prepared for surgery.

The Risk Score and Classification System here disclosed ends with the act of performing surgery on the patient, act which is only performed when a final positive recommendation is reached by the algorithm, and informed consent is obtained from the patient.

Lastly, not all surgeries are the same, and, consequently, different considerations have to be taken into account to define whether a particular surgery is safe for a particular patient. The following table shows how these factors are to be taken into account to define the best course of action:

TABLE 11
Algorithm Recommendations Table for surgery type

| Risk | | | Combination | |
|---|---|---|---|---|
| Level | Health Conditions | Allowed Surgeries | Logic | Surgery Type (Group) |
| Low (1.0 | No additional risk | All surgeries (up to 6 | Allowed up | Group 1: Rhinoplasty, |
| to <1.2) | factors | hours) | to 5 hours | Blepharoplasty, Lip Lift, |
| | | | | Facial Fat Grafting etc. |
| | | | | Group 2: |
| | | | | Abdominoplasty, Body |
| | | | | Lift Full Facelift etc. |
| Low (1.0 | Smoker | Surgeries up to 5 | Allowed up | Group 1: Rhinoplasty, |
| to <1.2) | | hours without large | to 4 hours | Otoplasty, Mini |
| | | skin undermining | | Abdominoplasty, etc. |
| Low (1.0 | Compensated | Surgeries up to 4 | Allowed up | Group 1: |
| to <1.2) | Hypertension | hours with cardiologic | to 4 hours | Blepharoplasty, Lip Lift |
| | | monitoring | | Otoplasty, etc. |
| Low (1.0 | BMI 25-30 | Surgeries up to 5 | Allowed up | Group 2: |
| to <1.2) | | hours | to 4 hours | Abdominoplasty, Body |
| | | | | Lift, |
| | | | | Lipoabdominoplasty, |
| | | | | etc. |
| Moderate | No additional risk | Surgeries up to 5 | Allowed up | Group 2: Body Lift, |
| (1.2 to | factors | hours | to 4 hours | Lipoabdominoplasty, |
| 1.4) | | | | Full Facelift, etc. |
| Moderate | Smoker | Surgeries up to 4 | Allowed up | Group 1: |
| (1.2 to | | hours without large | to 3 hours | Blepharoplasty, |
| 1.4) | | skin undermining | | Rhinoplasty, |
| | | | | Labiaplasty, etc. |
| Moderate | Compensated | Surgeries up to 4 | Allowed up | Group 1: Rhinoplasty, |
| (1.2 to | Hypertension | hours with cardiologic | to 3 hours | Liposuction, Otoplasty |
| 1.4) | | monitoring | | etc. |
| Moderate | BMI 25-30 | Surgeries up to 4 | Allowed up | Group 2: Mini |
| (1.2 to | | hours | to 3 hours | Abdominoplasty, Mini |
| 1.4) | | | | Facelift, etc. |
| High (1.4 | No additional risk | Surgeries up to 3 | No | Group 1: |
| to 2.5) | factors | hours up to 5 hours if | combinations | Blepharoplasty, |
| | | age >65 | allowed | Otoplasty, Rhinoplasty |
| | | | | Facial Fat Grafting etc. |
| High (1.4 | Smoker | No surgeries allowed | Not | N/A |
| to 2.5) | | | applicable | |
| High (1.4 | BMI >30 | Surgeries up to 2 | No | Group 1: |
| to 2.5) | Coagulation | hours | combinations | Blepharoplasty, |
| | Disorders | | allowed | Rhinoplasty, etc. |
| High (1.4 | Severe | Surgeries up to 1-2 | No | Group 1: Otoplasty, |
| to 2.5) | Cardiovascular | hours | combinations | Minor Facelift etc. |
| | Issues Smoker | | allowed | |

### Surgery Group Breakdown

Group 1 (less than 3 hours): Simple procedures:
Rhinoplasty
Blepharoplasty
Lip Lift.
Otoplasty
Facial Fat Grafting
Liposuction
Labiaplasty

Group 2 (between 3 to 5 hours): Medium complexity procedures:
Abdominoplasty
Mini Abdominoplasty
Full Facelift.
Body Lift
Lipoabdominoplasty

Group 3 (up to 6 hours): More complex procedures:
Circumferential Body Lift
Abdominoplasty+Breast Augmentation
Full Facelift with Neck Lift

### Clarification for Unlisted Surgeries:

The time for each surgery can be estimated for the purpose of this algorithm using time-estimation lists and methods already known in the field. If a specific surgery is not mentioned in the table (for example, lipotransference to pectorals), the algorithm prioritizes the filters in the following order:
1. Risk Level: The procedure is evaluated based on the patient's risk score (e.g., moderate risk).
2. Health Conditions: The patient's specific health conditions are considered (e.g., smoking, hypertension, BMI).
3. Surgery Duration: The procedure is categorized based on its estimated duration (e.g., surgery lasting less than 4 hours).

### Example for Lipotransference to Pectorals:

Risk Level: Moderate Risk
Health Condition: Smoker
Recommended Surgery: Lipotransference to Pectorals is homologated according to its duration (under 4 hours in this case).

Thus, the algorithm will recommend the surgery based on the input data and filters, prioritizing safety and risk management.

### Explanation of the Filters and How They Generate Recommendations

The disclosed algorithm operates with three core filters to generate personalized surgical recommendations based on the patient's health profile. Each step ensures safety by assessing the patient's risk and applying limitations accordingly.

### Step 1: Risk Score Filter

The first filter classifies patients into three risk categories based on their Risk Score, which is calculated by evaluating various patient-specific health factors such as:
Age.
BMI (Body Mass Index)
Smoking habits
Cardiovascular issues
Previous medical history

### Risk Categories:

Low Risk (1.0 to <1.2): These patients are considered to have minimal risks, allowing them to undergo a broad range of surgical procedures (up to 6 hours) with the possibility of combining surgeries.
Moderate Risk (1.2 to 1.4): These patients have moderate health risks, limiting them to surgeries of up to 5 hours, with some restrictions on combinations.
High Risk (1.4 to 2.5): These patients are at higher risk, restricting them to simpler surgeries (up to 3 hours), with no combinations allowed. In cases where the patient's only risk factor is age (>65), surgeries of up to 5 hours may be allowed.

### Step 2: Health Condition Filter

The second filter adjusts the recommendations based on specific health conditions that influence the patient's overall safety during surgery. These include:
Smoking: Patients who smoke are at higher risk of complications, particularly in surgeries involving large skin undermining, such as circumferential lipoabdominoplasty or body lift.
Low-risk smokers are limited to surgeries of up to 5 hours.
Moderate-risk smokers are limited to surgeries of up to 4 hours.
High-risk smokers are not allowed to undergo surgery.
Large skin undermining surgeries are strictly prohibited for smokers in any risk category.
Hypertension (Grade 1 or 2): Compensated hypertension under medication is manageable, but requires cardiologic monitoring during surgery.
Low and moderate-risk patients with compensated hypertension can undergo surgeries of up to 4 hours with cardiologic monitoring.
High-risk patients are restricted to shorter surgeries or not allowed to undergo surgery if the hypertension is severe.
BMI (Body Mass Index):
Patients with BMI 25-30 are considered to be at moderate risk.
Low-risk patients with this BMI can undergo surgeries up to 5 hours.
Moderate-risk patients with this BMI are limited to surgeries up to 4 hours.
High-risk patients with a BMI>30 are limited to surgeries of no more than 2 hours.

### Step 3: Combination of Surgeries

The final filter determines whether multiple procedures can be combined during a single surgical session. This filter ensures that combined surgeries do not pose additional risks by reducing the total surgery time and limiting the types of combinations:
Low-risk patients can combine procedures up to a total surgery time of 5 hours.
Moderate-risk patients can combine procedures up to a total surgery time of 4 hours.
High-risk patients are not allowed to combine procedures, as it increases the complexity and duration of the surgery, posing greater risks.

### Recommendation Generation Process Based on Patient Profile

The algorithm generates recommendations by following these steps:
1. Calculating the Risk Score:
   The algorithm first evaluates the patient's health profile, including BMI, smoking status, cardiovascular health, and age.
   Based on this assessment, the patient is assigned a Risk Score (1.0 to 2.5), which classifies them into low, moderate, or high-risk categories.
2. Applying Health Condition Filters:
   The next step is to check for additional health conditions, such as smoking, hypertension, or BMI. These conditions further limit the allowable surgeries based on the patient's risk score and specific health conditions.
   For example, a low-risk smoker can undergo surgeries up to 5 hours, but if the smoker is at high risk, they are not allowed to undergo surgery at all.
3. Evaluating Surgery Combinations:
   If the patient's health profile allows, the algorithm assesses whether multiple surgeries can be combined.
   Low and moderate-risk patients can combine surgeries with reduced total duration (up to 5 and 4 hours, respectively). High-risk patients are not permitted to combine procedures.
4. Generating the Final Recommendation:
   Based on the risk score, health conditions, and the possibility of combining surgeries, the algorithm generates a list of safe and recommended procedures for the patient according to their specific requirements.
   If no procedures are considered safe, the algorithm recommends that surgery be delayed or canceled, and the patient should focus on improving their health profile before reconsidering surgery.

### Example of How the Process Works:

Patient A: Low Risk (1.0), Non-Smoker, BMI 28
Allowed to undergo all surgeries up to 6 hours, including Rhinoplasty and Abdominoplasty.
Can combine surgeries, such as Rhinoplasty+Liposuction, for a total of 5 hours.
Patient B: Moderate Risk (1.3), Smoker, BMI 30.
Allowed to undergo smaller surgeries up to 4 hours, such as
Blepharoplasty or Otoplasty.
Cannot undergo large skin undermining surgeries, such as a Body Lift or Circumferential Lipoabdominoplasty.
Patient C: High Risk (1.8), Compensated Hypertension, Age 70
Allowed to undergo surgeries up to 3 hours, but up to 5 hours if age is the only risk factor.
Recommended procedure: Facial Fat Grafting and Blepharoplasty, up to a total of 3-5 hours.

Some general aspects of the present invention have been summarized so far in the first part of this detailed description and in the previous sections of this disclosure. Hereinafter, a detailed description of the invention as illustrated in the drawings will be provided. While some aspects of the invention will be described in connection with these drawings, it is to be understood that the disclosed embodiments are merely illustrative of the invention, which may be embodied in various forms. The specific materials, methods, structures, functional details, and scope disclosed herein are not to be interpreted as limiting. Instead, the intended function of this disclosure is to exemplify some of the ways—including the presently preferred ways—in which the invention, as defined by the claims, can be enabled for a Person of Ordinary Skill in the Art. Therefore, the intent of the present disclosure is to cover all variations encompassed within the spirit and scope of the invention as defined by the appended claims, and any reasonable equivalents thereof.

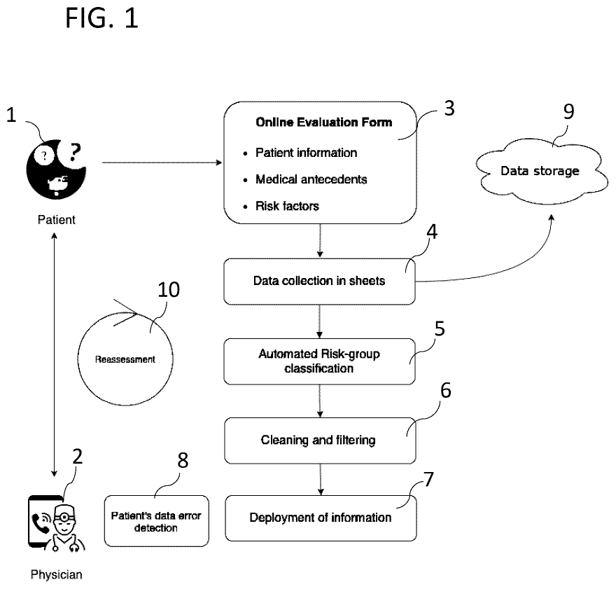

Referring to the drawings in more detail, FIG. 1 illustrates an embodiment of a system for establishing a predictive score for the risk of complications after plastic surgery in accordance with the present invention. In it, a Patient 1 introduces information to the system, which will be later retrieved by a Physician 2. This information is introduced by the Patient 1 through an Online Evaluation Form 3, which prompts the Patient 1 to submit self-assessed data about their personal information, medical antecedents, and risk factors, including all the information needed to calculate the Caprini Score of the patient as well as the cigarette consumption, BMI (or height and weight) and age of the patient, through an online form having checklists, multiple choice, dropdown menus radio buttons and/or similar formats for targeted data entry in which the relevant variables are considered.

The information collected through the Evaluation Form 3 can include items such as: Date, Email, Name, Last name, Age, BMI 36, Caprini Score, Price factor, Budget, Smoking (number of cigarettes consumed per week), Drugs, Alcohol, Venous thrombosis in legs or thighs (blood clot,) Diabetes, High blood pressure, Acute myocardial infarction, Ischemic heart disease, Vascular pathology, Cerebrovascular accident, Cardiac arrhythmia, Cancer, Thyroid, Allergies, Medications, Contraceptives (pills or patches), Inherited or family diseases (thrombosis, stroke, vasculopathy), Clotting or bleeding problems (with hematological diagnosis, e.g., factor deficit), previous surgery >1 month, Homocysteine, Surgery of interest, When would you like to have surgery?, Weight in Pounds, Height in inches, Pregnancies, Childbirth, C section, Number of abortions, Last menstruation date, Previous surgeries, Reoperation: The same surgery you require, Previous plastic surgeries, Asthma, Abdominal hernia, Stress in the last 3 months. It may be loss of a relative or job, separation, move, or economic factor, Covid 19 (any positive test), Gastric or duodenal ulcer (diagnosed by endoscopy), Chronic infectious diseases (Hepatitis B or C, HIV), Body Dysmorphic Disorder Questionnaire (BDDQ) based on DSM-IV diagnostic criteria for BDD, Are you overly concerned about the appearance of some part(s) of your body that you consider especially unattractive? Do these concerns preoccupy you? That is, do you think about them a lot and wish you could think about them less? Has your defect(s) caused you a lot of distress, torment, or pain? Has your defect(s) significantly interfered with your social life? Has your defect(s) significantly interfered with your schoolwork, your job, or your ability to function in your role? Are there things you avoid because of your defect(s)? How much time do you spend thinking about your defect(s) per day on average?≥1 h Is your main concern with your appearance that you aren't thin enough or that you might become fat? Times you breastfed, Number of previous breast surgery, Size of the implants you have or the closest number (only in case of previous surgery) Chest circumference measured in centimeters at the level of the nipples, Chest circumference measured in centimeters at the level of the nipples you want to have (which you want to increase approximately or bodice measurement), Circumference measured in centimeters of the chest at the level of the submammary sulcus, just below the breasts, Abdomen circumference at the level of the navel, measured in centimeters. Abdomen circumference at pubic level, measured in centimeters., Circumference just below the buttocks in the sub-gluteal groove, measured in centimeters Right Thigh: Circumference just below the buttocks, measured in centimeters, Left Thigh: Circumference just below the buttocks, measured in centimeters, Right Arm: Circumference just below the armpit, measured in centimeters, Left Arm: Circumference just below the left armpit, measured in centimeters.

This information is collected in sheets 4, being these sheets powered by Google Sheets, MySQL tables or any other software adequate for such end, and then stored in a database 9. An automated Risk-Group classification process 5 is then performed on the stored data. This process consists of applying the formula “Risk score=W₁f₁+W₂ f₂+W₃f₃+W₄f₄=1−2.5” to the information collected from the patient, where W₁ is the age of the patient, W₂ is the BMI of the patient, W₃ is the Caprini Score of the patient, W₄ is the number of cigarettes the patient smokes per week, f₁ is 100 \hat{ }(−3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more.

Subsequently, a process of Cleaning and Filtering 6 is performed and lastly, the information retrieved in the Deployment of Information step 7, in which the Physician 2 can access the processed data including the risk factor and all the main insights and statistics generated by the system. An algorithm of Patient data error detection is continuously performed throughout the process. Periodically, a Reassessment process 10 is also performed, whenever new patient information is available, there are data changes in patient data and/or in the risk factors' incidence levels or known correlations.

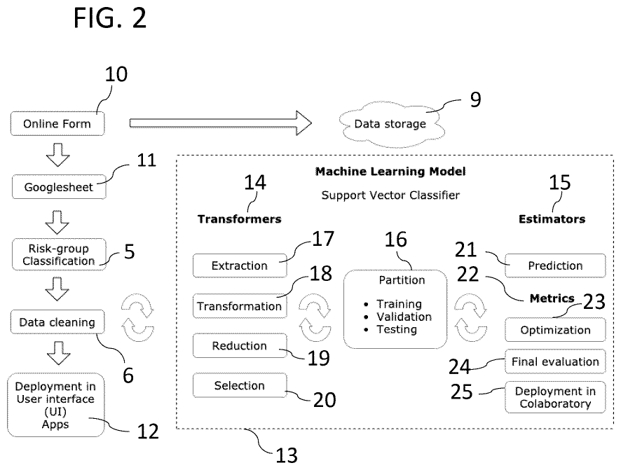

In FIG. 2, the Online Form 10 submits the collected data to a Google sheet 11 and stored in the data storage 9. The Risk Group classification 5 is performed based on this data which, after data cleaning 6, and deployed in a User Interface 12. A machine Learning model 13 is used for verifying the accuracy of the model and improving its predictive value based on the newly collected information. This model includes Transformers 14 and estimators 15. The transformers 14 include the extraction 17, Transformation 18, Reduction 19, and selection 20. A Partition 16 performs training, validation, and testing. The Estimators 15 include Prediction 21, the Metrics 22 used, optimization 23, final evaluation 24 and deployment in Collaboratory 25.

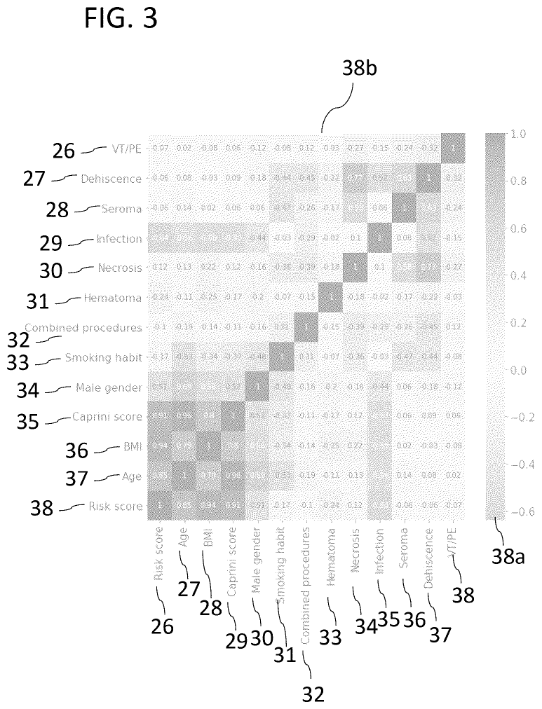

FIG. 3 shows the Patient's collected data on a table having the different risk factors both as columns and rows to check their correlation. The numbers 38b in the interior of the table correspond to the correlation between the factors in the corresponding column and row. A color scale factor 38a is also shown, showing the strength of the correlations by color according to their relevance, for an easier visualization. The clinical data considered are: VT/PE 26, Dehiscence 27, Seroma 28, Infection 29, Necrosis 30, Hematoma 31, Combined Procedures 32, Smoking habit 33, male gender 34, Caprini Score 35, BMI 36, age 37 and Risk Score 38.

The formula “Risk score=W₁ f₁+W₂f₂+W₃f₃+W₄f₄=1−2.5”, the variables selected being W₁ the age of the patient, W₂ the BMI of the patient, W₃ the Caprini Score of the patient, and W₄ the number of cigarettes the patient smokes per week, and the weighting factors being f₁ 100 \hat{ }(−3), f₂ 0.2 when W₂ is less than 25, 0.3 when W₂ is between 25 and 30, and 0.4 when W₂ is more than 30, f₃ 0.245, and f₄ 0.1 when W₄ is less than 7 and 0.3 when W₄ is 7 or more, have been developed as a conclusion of the studies performed with this method, for being highly relevant for risk prediction. However, variations in the variables used and their weighing factors, especially if obtained by the method hereby described, are encompassed within the spirit and scope of the present invention.

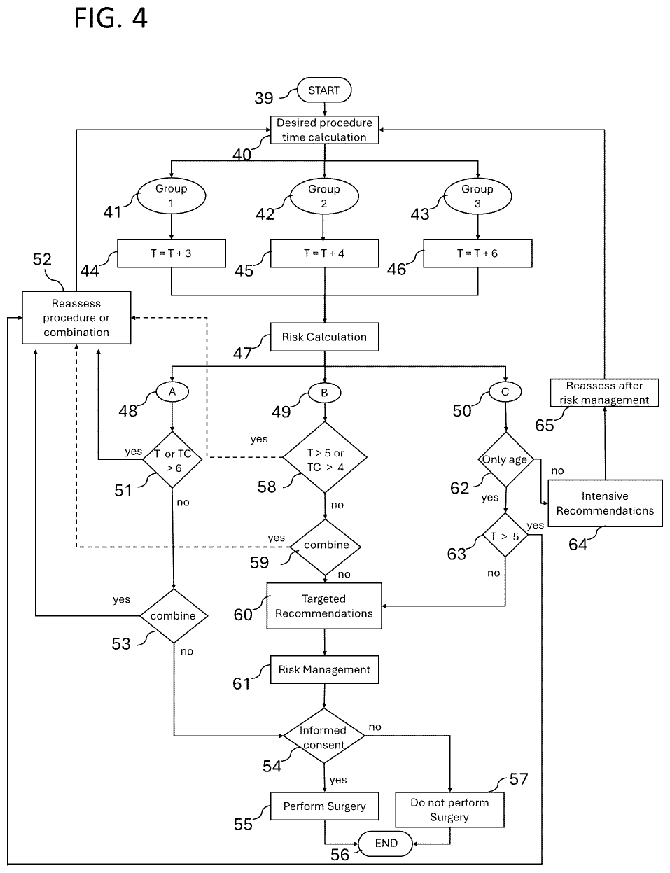

FIG. 4 is a flowchart showing the personalized recommendations of the algorithm, by assessing each patient's unique risk factors. The method starts at 39, with a calculation process 40 for calculating the time of surgery needed to perform the desired procedure. This can be calculated with steps 41 to 46 or alternatively by using any standard for time calculation accepted in the field. In any case, a variable for time (T) expressed in hours is defined, which represents how many hours of surgery are estimated for the desired procedure or procedures. In the shown embodiment, the data of paragraphs to of the present specification is used to classify the procedures in three groups: “Group 1” 41, which adds three hours to T in 44, (taking into account that T starts with a value of zero at the beginning, but it carries its previous value in the case of combined procedures, as will be shown later), “Group 2” 42, which adds four hours to T in 45, or “Group 3” 43, which adds six hours to T in 46. Once the value of T is defined, the Risk Calculation process 47 is performed as explained before, considering factors derived from a comprehensive analysis of their health data, such as age, BMI, blood pressure, medical history, and lifestyle habits (e.g., smoking). The risk score resulting from this Risk Calculation Process 47, calculated based on these variables, places the patient into one of three risk categories: A-Low Risk 48, B-Moderate Risk 49, or C-High Risk 50.

In the case of A-Low Risk 48 a time test 51 assesses whether the time T or the time of the combination TC exceed the six hours. If it does, the procedure(s) cannot be performed, as the maximum time allowed for surgeries, alone of combined, in the case of low risk is of six hours. The procedure or combination of procedures must be modified in 52, so that it takes no longer than six hours total, and then the process is resumed from the calculation process 40. If the time test 51 determines that the time T or the time of the combination TC do not exceed the six hours, step 53 questions whether a new procedure is desired to be added to the combination. If the answer is yes, the combination of procedures must be modified in 52 to the new desired combination and the time of combination TC of the new combination must be calculated starting over in 40. If the answer to step 53 is that no other procedure needs to be added, then the patient is informed of the risks associated with the surgery and asked for informed consent in step 54. If this consent is given by the patient, the surgery 55 is performed and the process ends 56. If this consent is not given by the patient, no surgery is performed 57 and the process ends 56.

Patients in the B-Moderate Risk 49 category typically have manageable but notable risks, such as elevated BMI or borderline high blood pressure. The algorithm tailors its recommendations to address these specific issues. For instance, it suggests daily blood pressure monitoring, consultations with a cardiologist, or a customized weight management plan in collaboration with a nutritionist, as described on Table 10. The algorithm considers the following question: Does the patient have any of the following conditions?
Smoker→Apply smoking restrictions (limit surgery time or exclude large skin undermining procedures).
Hypertension (compensated, Grade 1 or 2)→Limit surgery to less than 4 hours and require cardiologic monitoring.
BMI (25-30)→Adjust surgery time based on risk level.
Age>65→Special consideration, allowing surgery up to 5 hours for high-risk patients if no other risk factors exist.

A time test 58 assesses whether the time T exceeds five hours or the time of the combination TC exceeds four hours. If any of these is true, the procedure(s) cannot be performed, as the maximum time allowed for single surgeries in the case of moderate risk is of five hours, or of four hours in the case of combinations. The procedure or combination of procedures must be modified in 52, so that it takes no longer than five hours or four hours total for combinations, and then the process is resumed from the calculation process 40. If the time test 58 determines that the time T does not exceed five hours or the time of the combination TC does not exceed four hours, step 59 questions whether a new procedure is desired to be added to the combination. If the answer is yes, the combination of procedures must be modified in 52 to the new desired combination and the time of combination TC of the new combination must be calculated starting over in 40. If the answer to step 59 is that no other procedure needs to be added, targeted recommendations 60 are made according to Table 10, and the appropriate risk management 61 is performed on the patient. Then the patient is informed of the risks associated with the surgery and asked for informed consent in step 54. If this consent is given by the patient, the surgery 55 is performed and the process ends 56. If this consent is not given by the patient, no surgery is performed 57 and the process ends 56.

For patients in the C-high-risk 50 category, an “only age” 62 question is asked by the algorithm. This question assesses whether the reason for the risk being considered high is exclusively due to the age of the patient (older than sixty-five years old) not having other risk factors present. If this is true, that is to say, if the only reason for high-risk classification is the age of the patient, a time test 63 assesses whether the time T exceeds five hours. (No combinations are allowed for high-risk patients). If it does, the procedure cannot be performed, as the maximum time allowed for single surgeries in the case of high risk is of five hours. The procedure must be modified in 52, so that it takes no longer than five hours total, and then the process is resumed from the calculation process 40. If the time test 63 determines that the time T does not exceed five hours, targeted recommendations 60 are made according to Table 10, and the appropriate risk management 61 is performed on the patient. Then the patient is informed of the risks associated with the surgery and asked for informed consent in step 54. If this consent is given by the patient, the surgery 55 is performed and the process ends 56. If this consent is not given by the patient, no surgery is performed 57 and the process ends 56. If the answer to the “only age” 62 question is “no”, intensive recommendations 64 are performed on the patient based on Table 10, to try to improve the general health of the patient to ready them for surgery. These may include advanced diagnostic tests like ergometry or Doppler ultrasound to assess cardiovascular health, or consultations with specialists such as hematologists or endocrinologists. The goal is to thoroughly evaluate and mitigate multiple, significant risk factors before surgery. After risk management, a reassessment 65 is made, where the process is resumed from the calculation process 40 to determine if the risk group of the patient has now changed.

Finally, it should be noted that the above descriptions are based on data at the time of the invention, are dynamic, and are not intended to limit the invention. Especially the development of user interfaces, for example, applications and user interfaces that will change according to technological evolution, as well as medical recommendations that will change based on scientific evidence. Although the invention has been described in detail with reference to the above embodiments, it is still possible that the technical solutions described above will be modified, according to patient's needs and preferences, and technological advances. For example, the use of the algorithm and system itself is not limited to plastic surgery and can be adapted to all surgical specialties. Any modification, replacement, improvement, etc. conducted within the spirit and principle of the invention will be included in the scope of protection of the invention.

1. A system for performing low-risk plastic surgery on a patient, said system comprising a computing system for establishing a predictive score for the risk of complications after plastic surgery, and a personalized recommendations system, said computing system comprising:
a. a patient interface;
b. a physician interface;
c. an online evaluation form accessible through said patient interface, said online evaluation form comprising a plurality of input-form-fields to enter information about a patient, said information comprising data needed to calculate a Caprini Score, cigarette consumption, BMI, and age of the patient;
d. a database in which said information is collected, stored, and from which said information can be retrieved;
e. an automated risk score calculation algorithm based on said information, using the following a risk score formula to calculate a Risk Score of the patient, said risk score formula being:
$$Risk Score = W 1 f 1 + W 2 f 2 + W 3 f 3 + W 4 f 4$$
Where:
W1, W2, W3, and W4 are variables of the formula
f1, f2, f3, and f4 are factors of the formula said variables and factors defined as:
W1 is “age of the patient”, W2 is “BMI of the patient”, W3 is “Caprini Score of the patient”, W4 is “number of cigarettes the patient smokes per week”, f1 is “100 \hat{ }(−3)”, f2 is “0.2” when W2 is less than 25, “0.3” when W2 is between 25 and 30, and “0.4” when W2 is more than 30, f3 is “0.245”, and f4 is “0.1” when W4 is less than 7 and “0.3” when W4 is 7 or more; and
f. a risk classification algorithm to assign a Risk Group to the patients based on said Risk Score, as “low risk” (when Risk Score is 1 to <1.2), “moderate risk” (when Risk Score is ≥1.2 to <1.4), and “high risk” (when Risk Score is ≥1.4) of complications;
wherein said computing system is embodied in a computer having a processor, a memory, a screen, and internet access;
wherein said personalized recommendations system provides a final decision based on said risk score and said variables W1, W2, W3, and W4, by following an algorithm recommendations table;
and wherein surgery is performed on the patient only when said final decision is positive and the patient gives informed consent.

2. The system for performing low-risk plastic surgery on a patient of claim 1, further comprising a deployment of information process to display said risk score and said risk group through said Physician's interface.

3. The system for performing low-risk plastic surgery on a patient of claim 1, further comprising a system for fine-tuning said variables and said factors, by collecting clinical data, performing statistical analysis of data, using Pearson's correlation coefficient test and p-value to find risk factors with statistically significant correlation with complications, performing a Pearson correlation coefficient test and a p-value between the risk score and the risk factors to determine which are independent predictors, establishing a predictive scoring system based on a multifactorial correlation analysis, as a Coefficient of multiple correlation, and validating said automated risk score calculation algorithm by machine learning using a vector classification system (VCS).

4. A method for establishing a scoring system and classification into risk groups for predicting the risk of complications after plastic surgeries comprising the steps of:
(1) Collecting information from a patient, said information comprising data needed to calculate a Caprini Score of the patient as well as cigarette consumption, BMI, and age of said patient, through an online evaluation form;
(2) Storing the information from step 1 on a database;
(3) Calculating the Caprini Score of the Patient;
(4) Calculating a Risk Score of the patient by applying a risk score formula, said risk score formula being “Risk score=W₁f₁+W₂f₂+W₃f₃+W₄f₄=1−2.5” to the information collected from the patient in step 1, where W₁ is “age of the patient”, W₂ is “BMI of the patient”, W₃ is “Caprini Score of the patient”, W₄ is “number of cigarettes the patient smokes per week”, f₁ is 100 \hat{ }(−3), f₂ is 0.2 when W₂ is less than 25, 0.3 when W₂ is between 25 and 30, and 0.4 when W₂ is more than 30, f₃ is 0.245, and f₄ is 0.1 when W₄ is less than 7 and 0.3 when W₄ is 7 or more;
(5) Classifying Patients in Risk Groups where three different risk groups are defined, said risk groups being: low risk when the Risk score is (1 to <1.2), moderate risk when the Risk score is (≥1.2 to <1.4), and high risk when the Risk score is (≥1.4);
(6) displaying the Risk Score calculated on step 4 and the risk group calculated on step 5, together with the information collected on step 1, through a Physician's interface in a computer or electronic device comprising a processor and a memory and connected to the Internet;
(7) reaching a final decision by considering said risk score and said variables W1, W2, W3, and W4, by following an algorithm recommendations table; and
(8) performing surgery on the patient only when said final decision is positive and the patient gives informed consent.
